(19) United States
(12) Patent Application Publication
(10) Pub. No.: US 2025/0191761 A1
(43) Pub. Date: Jun. 12, 2025

(54) Methods and Systems for Refining a Diagnostic Machine-Learning Model

(71) Applicant: IDEXX Laboratories, Inc., Westbrook, ME (US)

(72) Inventors: David Steines, Westbrook, ME (US); John McGibbon, Westbook, ME (US)

(21) Appl. No.: 18/958,832

(22) Filed: Nov. 25, 2024

Related U.S. Application Data
(60) Provisional application No. 63/606,988, filed on Dec. 6, 2023.

(51) Int. Cl. G16H 50/20; G16H 50/70

(52) U.S. Cl. CPC G16H 50/20; G16H 50/70

(57) ABSTRACT

An example method for refining a diagnostic machine-learning model includes receiving, at a central computing device and from multiple point-of-care (POC) analyzers of a plurality of POC analyzers, respective signals indicative of user input approving use of the multiple POC analyzers as remote training nodes, sending an initial training model to the multiple POC analyzers, at least some of the multiple POC analyzers training the initial training model on respective remote data sets stored on the at least some of the multiple POC analyzers using respective processors of the at least some of the multiple POC analyzers to form revised training models, receiving the revised training models from the at least some of the multiple POC analyzers, averaging parameters of the revised training models based on configurable criteria to form an adjusted training model, and sending the adjusted training model to the plurality of POC analyzers.

### CROSS-REFERENCE TO RELATED APPLICATION

The present disclosure claims priority to U.S. application No. 63/606,988, filed on Dec. 6, 2023, the entire contents of which are herein incorporated by reference.

### FIELD

The present disclosure relates generally to methods and systems for refining a diagnostic machine-learning model through use of multiple point-of-care (POC) analyzers, each of which stores a respective remote data set, as remote training nodes and averaging parameters of revised training models received from the POC analyzers to form an adjusted training model for use by the POC analyzers.

### BACKGROUND

Artificial intelligence and machine learning model training requires access to (1) significant computing power, and (2) significant amounts of test result data (e.g., training data). Fortunately, massive volumes of data are collected every day by large number of entities such as research centers, veterinaries, hospitals, or other medical entities. Analysis of the data could improve learning models.

A problem of training the learning models could be solved by distributed computing by taking advantage of resource storage, computing power, cycles, content, and bandwidth of available devices. In such a distributed machine learning scenario, the datasets are transmitted to the multiple devices, and the devices solve a distributed optimization problem to collectively train the learning model. For distributed computing, similar (or identical) datasets are typically allocated to the multiple devices, which are then able to solve a problem in parallel. However, access to large, diverse healthcare datasets remains a challenge due to regulatory concerns over sharing protected healthcare information.

As such, privacy and even connectivity concerns may prohibit data from being shared between entities preventing largescale distributed methods. Additionally, using the same datasets to train the learning model across multiple devices lacks a diverse training of the model over time.

### SUMMARY

In an example, a method for refining a diagnostic machine-learning model is described, and the method comprises receiving, at a central computing device and from multiple point-of-care (POC) analyzers of a plurality of POC analyzers, respective signals indicative of user input approving use of the multiple POC analyzers as remote training nodes. Each respective POC analyzer of the plurality of POC analyzers comprises a processor and a memory storing a remote data set comprising medical data associated with patient samples analyzed by the respective POC analyzer. The method also comprises sending an initial training model to the multiple POC analyzers, and at least some of the multiple POC analyzers training the initial training model on respective remote data sets stored on the at least some of the multiple POC analyzers using respective processors of the at least some of the multiple POC analyzers to form revised training models. The method also comprises receiving, at the central computing device, the revised training models from the at least some of the multiple POC analyzers, averaging, by the central computing device, parameters of the revised training models based on configurable criteria to form an adjusted training model, and sending, by the central computing device, the adjusted training model to the plurality of POC analyzers.

In another example, a system configured to refine a diagnostic machine-learning model is described, and the system comprises a plurality of POC analyzers and each respective POC analyzer of the plurality of POC analyzers comprises a processor and a memory storing a remote data set comprising medical data associated with patient samples analyzed by the respective POC analyzer. Each respective POC analyzer is configured to train an initial training model on respective remote data sets stored on the respective POC analyzer using a respective processor of the respective POC analyzer. The system also comprises a central computing device in communication with the plurality of POC analyzers, and the central computing device is configured to: receive from multiple point-of-care (POC) analyzers of a plurality of POC analyzers, respective signals indicative of user input approving use of the multiple POC analyzers as remote training nodes, send the initial training model to the multiple POC analyzers, and receive revised training models from at least some of the multiple POC analyzers. The revised training models having been trained on respective remote data sets stored on the at least some of the multiple POC analyzers using respective processors of the at least some of the multiple POC analyzers. The central computing device is also configured to average parameters of the revised training models based on configurable criteria to form an adjusted training model, and send the adjusted training model to the plurality of POC analyzers.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The illustrative examples as well as a preferred mode of use, further objectives and descriptions thereof, will best be understood by reference to the following detailed description of an illustrative example of the present disclosure when read in conjunction with the accompanying drawings, wherein:

FIG. 1 illustrates an example of a system, according to an example implementation.

FIG. 2 illustrates an example of one of the POC analyzers in FIG. 1, according to an example implementation.

FIG. 3 illustrates an example of the central computing device in FIG. 1, according to an example implementation.

FIG. 4 is workflow diagram of an example process of functionality and messaging performed by components of the system of FIG. 1 to schedule and coordinate training of the initial training model by the POC analyzers, according to an example implementation.

FIG. 5 shows a flowchart of an example of a method for refining a diagnostic machine-learning model, according to an example implementation.

### DETAILED DESCRIPTION

Disclosed examples will now be described more fully hereinafter with reference to the accompanying drawings. Indeed, several different examples may be described and should not be construed as limited to the examples set forth herein. Rather, these examples are described so that this disclosure will be thorough and complete and will fully convey the scope of the disclosure to those skilled in the art.

Within examples described herein, technical solutions are provided to utilize point-of-care (POC) analyzers as remote training nodes to train a model for a machine learning algorithm using data sets stored locally on the POC analyzer as the training data. Diagnostic instruments, or more generally, POC analyzers, sit idle in clinics for portions of day. Thus, there is much available computing power. In an example including about 20,000 instruments in the field, a total compute power is approximately 36 petaflops, which is approximately 50% of computing performance of largest existing supercomputers. The idle computing power of POC analyzers is selectively utilized and leveraged for development, training, and testing activities by implementing a system that considers the POC analyzers as a network of remote training nodes.

Within examples described herein, several potential benefits include cost savings having training of models performed by deployed instruments (rather than cloud providers), higher quality and accelerated model improvements using more available data to train and retrain models where data remains on-instrument for retraining, accelerated time to field for the models due to reduced training times, and differential privacy learning is enabled by having one way transmissions to train models that use private information stored locally. For example, if two hospitals have private information, the model can be trained in each hospital separately and then averaged. This enables both hospitals to provide use of use their private data, and to contribute to the model without having to share data with each other.

Distributed computation and training of the model across the network of instruments supports a variety of compute patterns, such as retraining machine learning models using federated learning where some of the training data is stored on each remote node and models are trained on each node and then subsequently averaged at a central computing device, or a map-reduce scenario taking advantage of parallelism afforded by the large network of instruments enabling highly parallel (˜20,000 node) jobs. Furthermore, the models can be field tested on data stored locally on instruments. In an example, models are tested silently on data that is cached locally on instruments, enabling more rigorous and extensive model testing, as well as identification of challenging edge cases.

An example workflow includes a central computing device pushing a model to each instrument, a training client detects that the instrument is idle and launches a training job, the model is trained on data stored locally on each instrument, models are averaged by sending them back to the central computing device, and an updated model is evaluated for deployment. Averaging models may be simple averaging or weighted, and the training process may use supervised, semi-supervised, or unsupervised learning, for example. The training process is isolated from production routines on each instrument and is preempt-able. In this setup, some number of nodes can fail and that is okay because each node only trains on a small fragment of the data.

Implementations of this disclosure thus provide technological improvements that are particular to computer technology, for example, those concerning training a machine learning model on a local computing entity using data stored on the local computing entity. Computer-specific technological problems, such as selectively identifying instruments available for use as remote training nodes, can be wholly or partially solved by implementations of this disclosure. For example, implementation of this disclosure allows for the central computing device to use any number of criteria for selecting specific instruments in the field for use as remote training nodes, such as based on an amount and type of data stored locally on the instrument that will be used as training data.

The systems and methods of the present disclosure further address problems particular to generating training data for machine learning algorithms. For example, the central computing device optionally instructs instruments to use certain equipment settings during diagnostic testing in order to generate results for use as training data that are part of an overall strategy to gain a diverse set of training data from a group of instruments. Implementations of this disclosure can thus introduce new and efficient improvements in the ways in which training data is generated and in which models are trained for machine learning algorithms.

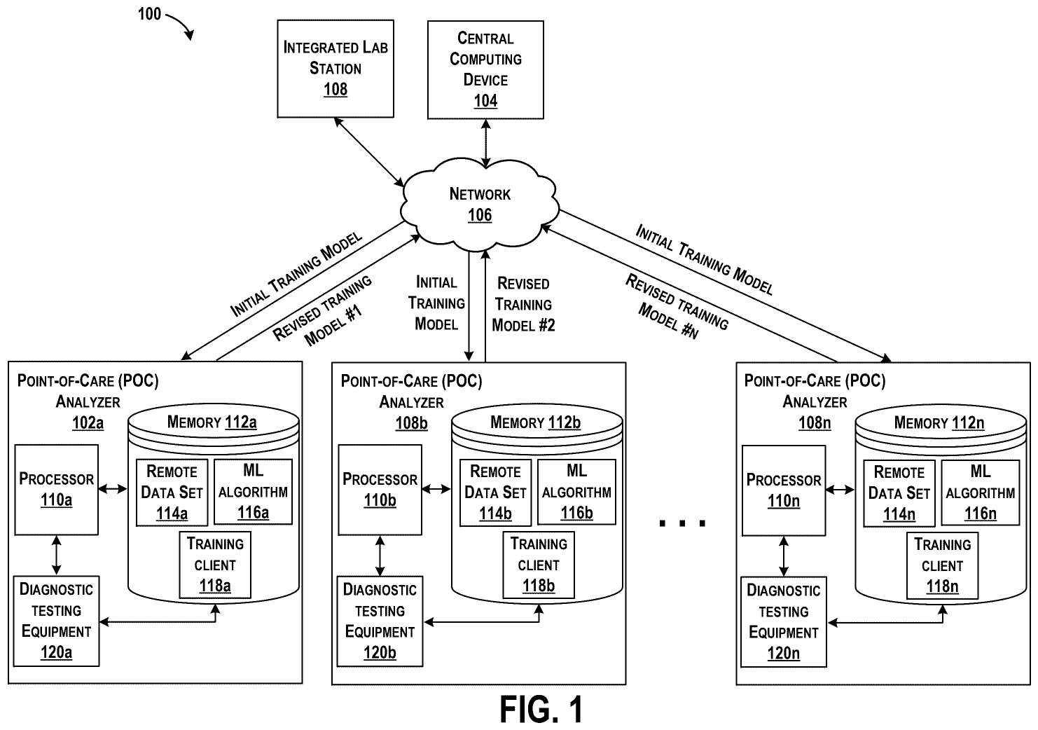

Referring now to the figures, FIG. 1 illustrates an example of a system 100, according to an example implementation. The system 100 includes a plurality of point-of-care (POC) analyzers 102a-n and a central computing device 104 in communication with the plurality of POC analyzers 102a-n through a network 106.

In embodiments, each of the POC analyzers 102a-n resides at a veterinary clinic. Some of the POC analyzers 102a-n reside at a same veterinary clinic, and some of the POC analyzers 102a-n reside at separate and distinct veterinary clinics. As referred to herein, the term “veterinary clinic” includes any entity at which non-human animals receive medical care, and can include brick and mortar locations, mobile clinics, on-line virtual clinics, pop-up clinics, and the like.

In addition, while the example depicted in FIG. 1 includes the POC analyzers 102a-n, it should be understood that this is merely an example, and embodiments according to the present disclosure can include any suitable number of POC analyzers at any number of veterinary clinics.

In embodiments, the POC analyzers 102a-n are communicatively coupled to the central computing device 104, and are operable to perform diagnostic testing of veterinary patients, for example. Examples of the POC analyzers 102a-n include any one or combination of veterinary analyzers operable to conduct a diagnostic test of a sample of a patient (e.g., operable to determine hemoglobin amounts in a blood sample, operable to analyze a urine sample, and/or the like). Such veterinary analyzers include, for example and without limitation, a clinical chemistry analyzer, a hematology analyzer, a microscopic analyzer, a urine analyzer, an immunoassay reader, a sediment analyzer, a blood analyzer, a digital radiology machine, and/or the like. The POC analyzers 102a-n contain a primary processor as well as a number of co-processors, in some examples. The primary and co-processors may be central processing units, graphics processing units, or other computational units.

In one example, an integrated lab station 108 is in communication with the POC analyzers 102a-n via the network 106 and is operable to receive diagnostic information output from the POC analyzers 102a-n. The POC analyzers 102a-n outputs signals, such as signals indicative of diagnostic test results or other information, to the integrated lab station 108.

In the system 100, the network 106 (e.g., Internet) provides access to the central computing device 104 and the integrated lab station 108 for all network-connected components. In some examples, more components of the system 100 may be in communication with the network 106 to access the central computing device 104 and the integrated lab station 108. Communication with the central computing device 104 and the integrated lab station 108 and/or with the network 106 may be wired or wireless communication (e.g., some components may be in wired Ethernet communication and others may use Wi-Fi communication). In still further examples, the network 106 provides access for the central computing device 104 and the integrated lab station 108 to communicate with the POC analyzers 102a-n.

The POC analyzers 102a-n include respective processors 110a-n and memory 112a-n storing remote data sets 114a-n, machine learning algorithms 116a-n, and training clients 118a-n.

The POC analyzers 102a-n also each includes respective diagnostic testing equipment 120a-n. The diagnostic testing equipment 120a-n can take many forms, and is operable to generate the respective remote data sets based on analyzing patient samples. The diagnostic testing equipment 120a-n thus includes imaging components (e.g., cameras), illumination components (e.g., light sources), chemical components, etc. as appropriate per specific POC analyzer. The patient samples include biological samples received from a patient including tissue, fecal, urine, or blood samples, for example.

Thus, the remote data sets 114a-n comprise medical data associated with patient samples analyzed by the respective POC analyzers 102a-n. Each of the remote data sets 114a-n is thus different and unique based on being output from the diagnostic testing equipment 120a-n, which analyze different and unique patient samples. Namely, different patient samples are analyzed by the different POC analyzers 102a-n resulting in different remote data sets.

The machine learning algorithms 116a-n refer to instructions executable by the processors 110a-n that use statistical models to generate outputs that rely on patterns and inferences by processing associated training data. Many types of machine learning algorithms can be used and are as classified into several categories. The machine learning algorithms 116a-n are executed to perform any number of different functions for diagnostic testing, clinical interpretations of test results, and providing follow-on recommendations, for example.

In supervised learning, the machine learning algorithms 116a-n build a mathematical model from a set of data that contains both the inputs and the desired outputs. The set of data is sample data known as the “training data”, in order to make predictions or decisions without being explicitly programmed to perform the task. For example, the machine learning algorithms 116a-n utilize the remote data sets 114a-n, respectively, as training data.

In another category referred to as semi-supervised learning, the machine learning algorithms 116a-n develop mathematical models from incomplete training data, where a portion of a sample input does not have labels. A classification algorithm can then be used when the outputs are restricted to a limited set of values.

In another category referred to as unsupervised learning, the machine learning algorithms 116a-n build a mathematical model from a set of data that contains only inputs and no desired output labels. Unsupervised learning algorithms are used to find structure in related training data, such as grouping or clustering of data points. Unsupervised learning can discover patterns in data, and can group the inputs into categories.

Alternative machine-learning algorithms may be used to learn and classify types of diagnostic test results to consider for generating the inquiries, such as deep learning though neural networks or generative models. Deep machine-learning may use neural networks to analyze prior test results through a collection of interconnected processing nodes. The connections between the nodes may be dynamically weighted. Neural networks learn relationships through repeated exposure to data and adjustment of internal weights. Neural networks may capture nonlinearity and interactions among independent variables without pre specification. Whereas traditional regression analysis requires that nonlinearities and interactions be detected and specified manually, neural networks perform the tasks automatically.

Still other machine-learning algorithms or functions can be implemented, such as any number of classifiers that receives input parameters and outputs a classification (e.g., attributes of the image). Support vector machine, Bayesian network, a probabilistic boosting tree, neural network, sparse auto-encoding classifier, or other known or later developed machine-learning algorithms may be used. Any semi-supervised, supervised, or unsupervised learning may be used. Hierarchal, cascade, or other approaches may be also used.

The machine learning algorithms 116a-n may thus be considered an application of rules in combination with learning from prior labeled data to identify appropriate outputs. Analyzing and relying on prior labeled data allows the machine learning algorithms 116a-n to apply patterns of diagnostic test results and associated outputs that are generally issued when such test results are observed, for example.

Thus, the machine learning algorithms 116a-n take the form of one or a combination of any of the herein described machine-learning algorithms, for example.

The training clients 118a-n of the POC analyzers 102a-n are executable by the processors 110a-n, respectively, to coordinate training of the machine learning algorithms 116a-n on an initial training model using the remote data sets 114a-n. In examples, the machine learning algorithms 116a-n use a model including parameters or weights, where the parameters or weights are trainable for use in different layers of the model. For instance, the model is trained/learned by adjusting its weights to minimize a cost of a particular objective function (such as a mean squared error between a POC analyzer output executing the machine learning algorithm with the model and an actual class label of a sample regarding a classification problem, for example).

Within examples, the training clients 118a-n are executable to control operation of the POC analyzers 102a-n to be in a diagnostic use mode where a patient sample is being analyzed via the diagnostic testing equipment 120a-n, or to be in a model training mode where processing power of the POC analyzers 102a-n is utilized to train a received training model using the remote data sets 114a-n. Generally, when the POC analyzers 102a-n are idle, the training clients 118a-n control operation of the POC analyzers 102a-n to be in a model training mode, or to be available to switch to the model training mode, for example.

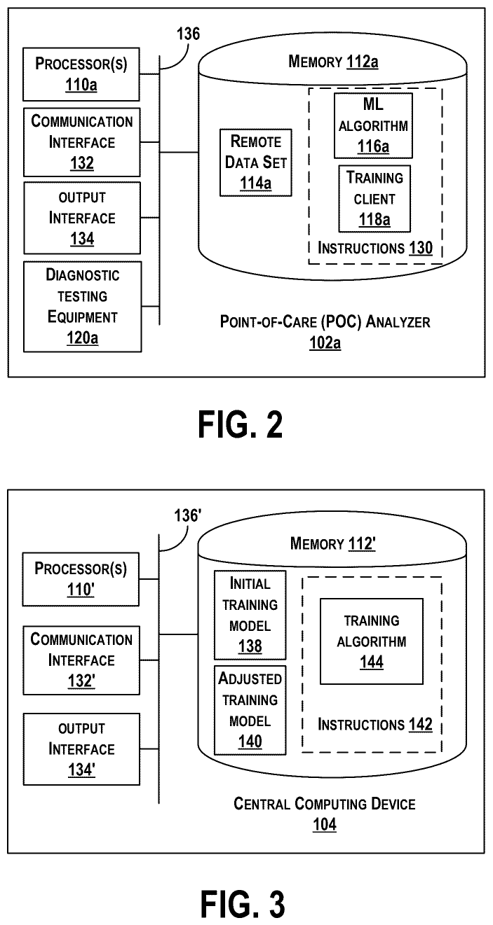

FIG. 2 illustrates an example of one of the POC analyzers 102a-n (e.g., the POC analyzer 102a) in FIG. 1, according to an example implementation. The POC analyzer 102a the includes the processor 110a, the memory 112a having stored therein instructions 130 that when executed by the processor 110a, causes the POC analyzer 102a to perform functions for diagnostic testing and updating and/or training models for machine learning algorithms, for example. The POC analyzer 102a in FIG. 2 is representative of any of the POC analyzers 102a-n.

To perform these functions, the POC analyzer 102a also includes a communication interface 132, an output interface 134, and each component of the POC analyzer 102a is connected to a communication bus 136. The POC analyzer 102a may also include hardware to enable communication within the POC analyzer 102a and between the POC analyzer 102a and other devices (not shown). The hardware may include transmitters, receivers, and antennas, for example. The POC analyzer 102a may further include a display.

In some embodiments, the communication interface 132 is a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 132 may be configured to receive input data from one or more devices, and may be configured to send output data to other devices.

The memory 112a includes or takes the form of non-transitory computer readable medium, such as one or more computer-readable storage media that can be read or accessed by the processor 110a. The memory 112a can include volatile and/or non-volatile storage components, such as optical, magnetic, organic or other memory or disc storage, which can be integrated in whole or in part with the processor 110a. In some examples, the memory 112a is implemented using a single physical device (e.g., one optical, magnetic, organic or other memory or disc storage unit), while in other examples, the memory 112a is implemented using two or more physical devices. The memory 112a thus is a computer readable storage, and the instructions 130 are stored thereon. The instructions 130 include computer executable code, and include the machine learning algorithm 116a and the training client 118a. The memory 112a also serves as data storage for the remote data set 114a.

The processor 110a may be general-purpose processors or special purpose processors (e.g., digital signal processors, application specific integrated circuits, etc.). The processor 110a receives inputs from the communication interface 132, and process the inputs to generate outputs that are stored in the memory 112a. The processor 110a is configured to execute the instructions 130 (e.g., computer-readable program instructions) that are stored in the memory 112a and are executable to provide the functionality of the POC analyzer 102a described herein.

The output interface 134 outputs information for transmission, reporting, or storage, and thus, the output interface 134 may be similar to the communication interface 132 and can be a wireless interface (e.g., transmitter) or a wired interface as well.

FIG. 3 illustrates an example of the central computing device 104 in FIG. 1, according to an example implementation. Some components of the central computing device 104 are substantially similar to or the same as components of the POC analyzer 102a within some examples, depending on where a function is programmed to be performed in a specific implementation. As a result, description of components within the central computing device 104 that are the same as the POC analyzer 102a are not repeated here, and such components include the processor 110′, the memory 112′, the communication interface 132′, the output interface 134′, and the communication bus 136′.

The memory 112′ of the central computing device 104 stores an initial training model 138, which includes weights and/or parameters of use by a machine learning algorithm, and an adjusted training model 140, which includes adjusted weights and/or parameters of use by the machine learning algorithm.

Within the memory 112′ of the central computing device 104, instructions 142 are stored for execution by the processor 110′ to perform functions of a training algorithm 144, which is executed to schedule and coordinate training of the initial training model 138 by the POC analyzers 102a-n.

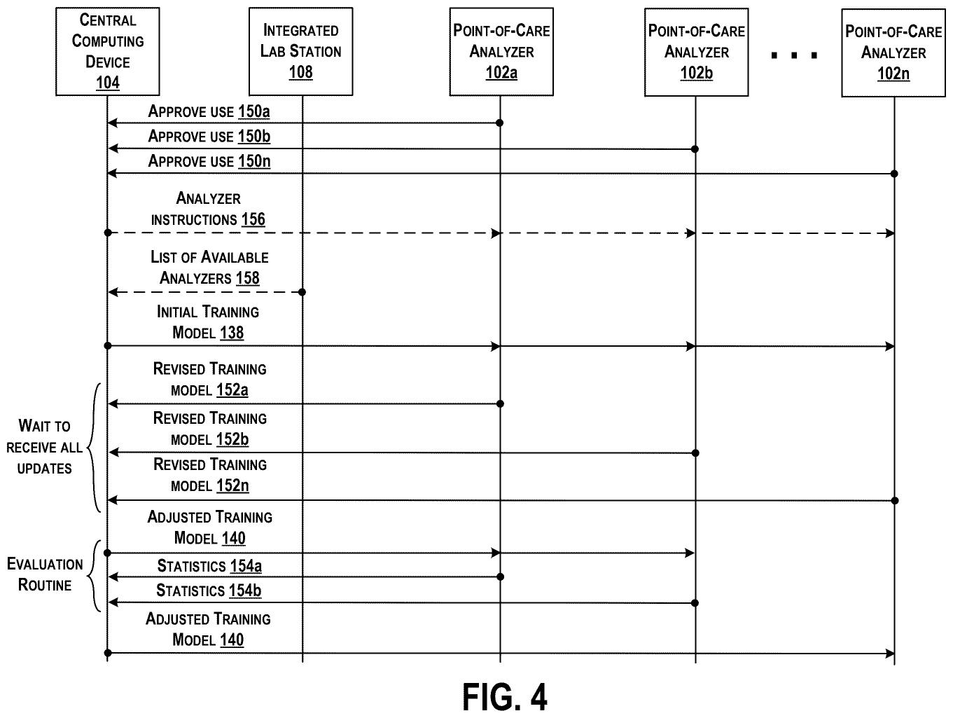

FIG. 4 is workflow diagram of an example process of functionality and messaging performed by components of the system 100 of FIG. 1 to schedule and coordinate training of the initial training model 138 by the POC analyzers 102a-n, according to an example implementation. The central computing device 104 schedules and coordinates training of the initial training model 138 by the POC analyzers 102a-n.

Initially, the central computing device 104 will receive from the POC analyzers 102a-n respective signals, referred to as approve use signals 150a-n, indicative of user input approving use of the POC analyzers 102a-n as remote training nodes. The central computing device 104 solicits use of the POC analyzers 102a-n, in some examples, prompting the response and opt-in approval by the POC analyzers 102a-n. In other examples, the POC analyzers 102a-n send the approval signals upon start-up or log-in by a user (e.g., without prompt by the central computing device 104).

In the workflow of FIG. 4, all of the POC analyzers 102a-n have opted in and sent the approval signals to the central computing device 104. However, in other examples, some or multiple of the POC analyzers 102a-n, but not all, will opt in and send approval signals.

The central computing device 104 will send the initial training model 138 to the POC analyzers 102a-n that have provided approval signals to be used as remote training nodes.

Each respective POC analyzer 120a-n is configured to train the initial training model 138 on respective remote data sets 114a-n stored on the respective POC analyzer 102a-n using respective processors 110a-n of the respective POC analyzer 102a-n. The respective remote data sets 114a-n remain stored on the POC analyzers 102a-n during training of the initial training model 138. As mentioned, each of the POC analyzers 102a-n generate the remote data sets 114a-n based on analyzing patient samples, which include biological samples received from a patient such as tissue or blood samples, and thus, the remote data sets 114a-n possibly include confidential patient or medical information.

The POC analyzers 102a-n train the initial training model 138 separately and individually to form individual revised training models. Each of the respective remote data sets 114a-n stored on the POC analyzers 102a-n contributes to a respective adjusted training model without sharing respective medical data between the POC analyzers 102a-n and without sharing the respective medical data with the central computing device 104.

Within examples, training of the initial training model 138 uses federated learning processes via computational processing power of respective processors 110a-n of the POC analyzers 102a-n and via the respective remote data sets 114a-n stored on the POC analyzers 102a-n. Training thus occurs on isolated environments.

A run-time for training the model is about 20 minutes, and the training clients 118a-n monitor usage of the POC analyzers 102a-n to ensure no conflict with usage of the processing power for training purposes versus usage of the processing power for diagnostic testing. Training will be scheduled and coordinated to occur during an idle time of the POC analyzers 102a-n. In one example, idle time of the POC analyzers 102a-n refers to a time period during which the diagnostic testing equipment 120a-n is not being utilized for analysis of a patient sample. Thus, based on any of the POC analyzers 102a-n receiving a request to analyze a patient sample using the diagnostic testing equipment 120a-n, the training clients 118a-n interrupt training of the initial training model 138. The diagnostic testing equipment 120a-n has priority for usage of processing power of the POC analyzers 102a-n. As a result, training can be interrupted at any time, and real-time results are not required or expected from the POC analyzers 102a-n.

The training client 118a-n assesses a convergence of the initial training model 138 to determine completion of the run-time. Once convergence is determined, model weights computed are packaged and sent back to the central computing device 104. Thus, the central computing device then receives revised training models 152a-n from the POC analyzers 102a-n, and each of the revised training models 152a-n has been trained on respective remote data sets 114a-n stored on the POC analyzers 102a-n using respective processors 110a-n of the POC analyzers 102a-n.

The central computing device 104 has a list of which POC analyzers are scheduled to train the initial training model 138, and waits to receive the computed weights and revised training models from them all. After receiving all of the revised training models 152a-n, the central computing device 104 averages parameters of the revised training models based on configurable criteria to form the adjusted training model 140. Configurable criteria include instructions for the POC analyzers to include or exclude data from the averaging procedure, a choice of an averaging procedure, a weighting of each POC analyzer output in the average, and parameters intrinsic to the training procedure, including but not limited to, learning rates or regularization terms, for example.

The newly computed model (i.e., the adjusted training model) is registered for logging and versioned, and then sent back out into the field to the POC analyzers. The adjusted training model 140 is a global model to be used globally by all POC analyzers, whereas the revised training models 152a-n are local models derived from at respective local sites.

In FIG. 4, the adjusted training model 140 is initially sent to only the POC analyzers 102a-b. In some examples, prior to sending the adjusted training model 140 to all of the POC analyzers 102a-n, the central computing device 104 sends the adjusted training model 140 to a subset of the POC analyzers 102a-n (such as the POC analyzers 102a-b) to be executed within an evaluation routine running on the subset of the POC analyzers using the remote data sets 114a-b stored on the subset of the POC analyzers. The POC analyzers 102a-b then send, and the central computing device 104 receives, statistics 154a-b from the subset of the POC analyzers related to execution of the adjusted training model 140. Based on the statistics 154a-b satisfying a threshold, the central computing device 104 sends the adjusted training model 140 to all of the POC analyzers 102a-n. Alternatively, based on the statistics 154a-b not satisfying the threshold, the central computing device 104 takes other action such as requesting retraining of the initial training model 138 by other POC analyzers.

Within examples, an evaluation routine runs on each of the POC analyzers 102a-n to execute the adjusted training model 140 on available diagnostic testing runs, which includes historical cached runs or new field data, to perform a comparison test of the adjusted training model 140 used on the different runs versus an existing model used on the different runs. Differences between outputs of the adjusted training model 140 and the existing model are analyzed to a mass statistics (e.g., deviations between outputs), which are then compared to a threshold. The threshold includes or considers sensitivities, specificities, precision, recall, area under the receiver operating characteristic, or other statistical measures. As an example, a threshold for an acceptable sensitivity is 90%, and the procedure would have to demonstrate it meets at least 90% sensitivity to be activated. Thresholds would be predetermined based on medical acceptance criteria.

Further feedback from pathologists can also be received to provide an additional manner to make a determination of which of the models (adjusted training model or existing model) is performing better over time.

Thus, the evaluation routine is useful as a verification for new models prior to distribution of the new models into the field. The statistics 154a-b are evaluated for threshold changes as compared with outputs of existing models to verify that anticipated changes are seen in results from use of the new models. As one example, when the initial training model 138 is developed, criteria for evaluation of outputs to compare to outputs of existing models is determined. Statistically, improvement can be expected to be observed over a thousand runs, and now, evaluating the adjusted training model 140 in the field enables advantageous use of real-world data that has not been part of a training set used for development.

In the workflow of FIG. 4, and optional messaging from the central computing device 104 to the POC analyzers 102a-n is shown as providing analyzer instructions 156. When a POC analyzer is analyzing a given run, specific exposure times, settings, etc. are used to evaluate patient samples in order to generate the outputs stored as remote data sets. In the example of optional messaging, the central computing device 104 sends the analyzer instructions 156 to the POC analyzers 102a-n that include instructions to set exposure times or other settings on the diagnostic testing equipment 120a-n in order to generate outputs differently. As a result, the remote data sets 114a-n will be unique and differentiated from each other due to analyzing different patient samples and due to using different settings of the diagnostic testing equipment 120a-n. The central computing device 104 thus controls how remote data sets are generated as well as training of the initial training model 138 using the generated remote data sets.

In a further optional message shown in the workflow of FIG. 4, the integrated lab station 108 stores paired test results of the remote data sets from the POC analyzers 102a-n, and the central computing device 104 queries the integrated lab station 108 to determine whether a paired test result exists for the remote data sets from the POC analyzers 102a-n. The integrated lab station 108 responds by sending a list of available analyzers 158 that include the requested information, and the central computing device 104 send the initial training model 138 to the POC analyzers 102a-n based on the received list. This example is valuable in a case where multimodal training occurs, for example, when pairing a hematology analyzer with a microscopy analyzer. As an example, the hematology analyzer contains a ground truth such as a number of a certain type of blood cell, and a microscope is trained to identify that type of blood cell knowing proportions that are likely present in the image. A strength of the hematology analyzer output is the large number of cells, and hence, well-powered statistics it provides, while the microscopy system is able to create a high-resolution, detailed view of the blood cell. There exist many embodiments where analyzers may be paired.

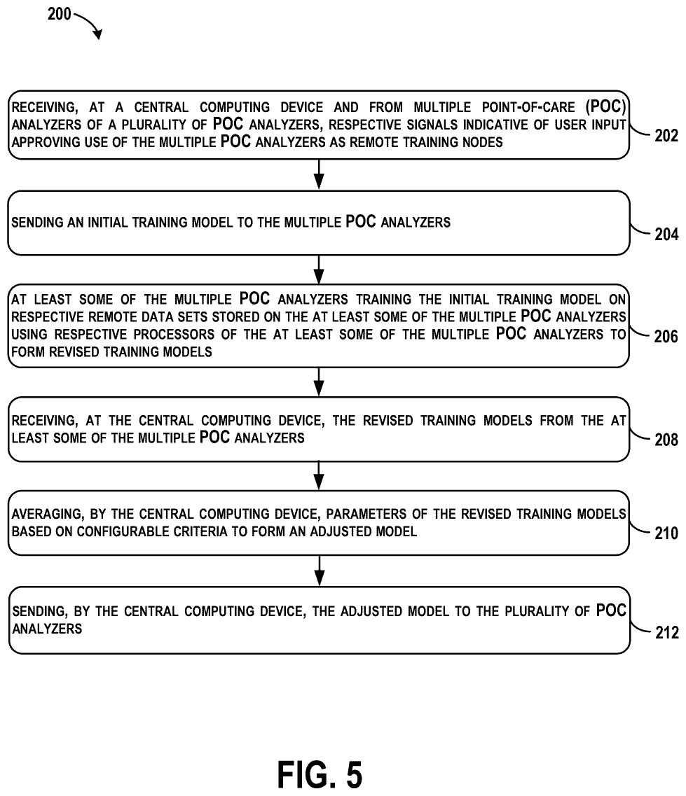

FIG. 5 shows a flowchart of an example of a method 200 for refining a diagnostic machine-learning model, according to an example implementation. Method 200 shown in FIG. 5 presents an example of a method that could be used with the system 100 shown in FIG. 1, the central computing device 104, shown in FIG. 1, or the POC analyzers 102a-n shown in FIG. 1, for example. Further, devices or systems may be used or configured to perform logical functions presented in FIG. 5. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-212. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 5, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Some functions of the method 200 include receiving and sending of communications/data between components, and reference is made to the system 100 in FIG. 1 where communications occur via the network 106, for example.

At block 202, the method 200 includes receiving, at the central computing device 104 and from multiple point-of-care (POC) analyzers of a plurality of POC analyzers 102a-n, respective signals indicative of user input approving use of the multiple POC analyzers as remote training nodes, and each respective POC analyzer of the plurality of POC analyzers comprises a processor and a memory storing a remote data set comprising medical data associated with patient samples analyzed by the respective POC analyzer. In an example, the method 200 includes at least some of the multiple POC analyzers generating the respective remote data sets based on analyzing the patient samples, and the patient samples comprise biological samples received from a patient including tissue or blood samples.

At block 204, the method 200 includes sending an initial training model to the multiple POC analyzers.

At block 206, the method 200 includes at least some of the multiple POC analyzers training the initial training model on respective remote data sets stored on the at least some of the multiple POC analyzers using respective processors of the at least some of the multiple POC analyzers to form revised training models. In an example, the respective remote data sets remain stored on the at least some of the multiple POC analyzers during training of the initial training model. In addition, the method 200 also includes, based on any of the at least some of the multiple POC analyzers receiving a request to analyze a patient sample, interrupting training of the initial training model.

In further examples, the method 200 includes training the initial training model separately by the at least some of the multiple POC analyzers to form the revised training models, and each of the respective remote data sets stored on the at least some of the multiple POC analyzers contributes to the adjusted training model without sharing respective medical data between the plurality of POC analyzers and without sharing the respective medical data with the central computing device.

In one example, block 206 includes training the initial training model using federated learning via computational processing power of respective processors of the at least some of the multiple POC analyzers and via the respective remote data sets stored on the at least some of the multiple POC analyzers.

At block 208, the method 200 includes receiving, at the central computing device 104, the revised training models from the at least some of the multiple POC analyzers.

At block 210, the method 200 includes averaging, by the central computing device 104, parameters of the revised training models based on configurable criteria to form an adjusted training model.

At block 212, the method 200 includes sending, by the central computing device 104, the adjusted training model to the plurality of POC analyzers.

In some examples, the method 200 also includes prior to sending the adjusted training model to the plurality of POC analyzers, sending the adjusted training model to a subset of the plurality of POC analyzers to be executed within an evaluation routine running on the subset of the plurality of POC analyzers using the remote data sets stored on the subset of the plurality of POC analyzers, receiving statistics from the subset of the plurality of POC analyzers related to execution of the adjusted training model, and based on the statistics satisfying a threshold, sending the adjusted training model to all of the plurality of POC analyzers.

The central computing device 104 in the method 200 of FIG. 5 sends the initial training model to the multiple POC analyzers that provided approved use signals as remote training nodes. In further examples, the central computing device 104 uses criteria for selecting which POC analyzers among a group of POC analyzers to use as remote training nodes. Various different criteria can be considered and examples are provided below.

In one example, the central computing device 104 determines which of the plurality of POC analyzers 102a-n are not currently being utilized to analyze a patient sample, and the central computing device 104 solicits use of POC analyzers of the plurality of POC analyzers 102a-n that are not currently being utilized to analyze the patient sample as respective remote training nodes. Thus, in response to determining that the multiple POC analyzers are not being utilized to analyze the patient sample, the multiple POC analyzers train the initial training model.

In another example, the central computing device 104 solicits use of POC analyzers of the plurality of POC analyzers 102a-n that are located in different geographic areas to gain access to geographically diverse remote data sets for training the initial training model. The POC analyzers 102a-n analyze patient samples that include non-human or animal patients, and animal patients located in different geographic area are likely to exhibit different characteristics resulting in diverse remote data sets.

In another example, the central computing device 104 solicits use of POC analyzers of the plurality of POC analyzers 102a-n that are of multiple different types of POC analyzers to gain access to medically diverse remote data sets for training the initial training model. Such selections enable a potential to draw in information from other analyzers, such as hematology or other types of analyzers, for example.

In another example, the central computing device 104 accesses prior stored remote data sets of the plurality of POC analyzers to determine which of the plurality of POC analyzers 102a-n has desired or complete metadata for the medical data, and then the central computing device 104 solicits use of POC analyzers of the plurality of POC analyzers that have the desired or complete metadata for the medical data to gain access to complete remote data sets for training the initial training model. Complete metadata includes all fields of a medical report, for example, populated with data of the patient to include all demographic information of the patient. Complete metadata or desired data also includes additional information that a clinician enters per sample, such as full labels for all radiology shot-types, for example.

In still another example, each respective POC analyzer of the plurality of POC analyzers generates the remote data set based on analyzing the patient samples and the patient samples comprise biological samples received from a patient including tissue or blood samples, and the central computing device 104 queries the plurality of POC analyzers to determine an amount of data in the remote data set stored on each of the plurality of POC analyzers. The central computing device 104 then sends the initial training model to the multiple POC analyzers based on the amount of data in the remote data set stored on the multiple POC analyzers satisfying a threshold amount. An amount of data can ensure that the remote data set is populated with enough data to sufficiently train the model. Further information, such as an age of the data, or how the data was generated (e.g., whether the patient sample for analysis was collected at the clinic or at home), can also be used as criteria for selection of which POC analyzers to select for use as remote training nodes. As a result, type of remote data sets and qualify of the remote data sets are effective criteria to be used by the central computing device 104, in some examples.

In another example, the initial training model 138 relates to an algorithm executed on medical data related to a patient of a specific species, and the central computing device 104 solicits use of POC analyzers of the plurality of POC analyzers 102a-n that are utilized to analyze patient samples of the specific species to gain access to species dependent remote data sets for training the initial training model.

In another example, the diagnostic machine-learning model relates to a medical condition, and the central computing device 104 receives from one of the at least some of the multiple POC analyzers that returned a respective revised training model, a notification indicating that a subsequent patient sample from a repeat patient for the medical condition has been received. In this example, it is useful to gain further training of the model on the specific medical condition, and thus, once additional or new training data is present, the central computing device 104 sends the adjusted training model to the one of the at least some of the multiple POC analyzers that sent the notification, and the POC analyzer retrains the adjusted training model based on an updated remote data set including medical data resulting from analysis of the subsequent patient sample. It is useful in some instances where a patient is a repeat visitor at a same clinic and medical condition are monitoring to track when a patient has presented a subsequent sample in order to retrain the model on the newly generated remote data set.

Still further, the central computing device 104 implements a scheduler, in some examples, so as to utilize POC analyzers as remote training nodes during times of non-usage patterns, so as to schedule processing loads to run when the POC analyzers are expected to be idle (e.g., overnight).

Within examples described herein, the POC analyzers are utilized, in selective ways, to train models for use in machine learning algorithms using data stored locally on each POC analyzer. This provides the benefit that the data remains locally on each analyzer providing privacy benefits, and provides an ability to train the model on greater diversity of data. Example systems includes thousands of analyzers in the field, and so the central computing device 100 can be selective of which analyzer to use as a remote training node to gain a better representation of diversity in population, and then the localized training is aggregated to leverage variations seen in the field.

With reference to FIGS. 2-3, and throughout the disclosure, some components are described as “modules,” and such components include or take a form of a general purpose or special purpose hardware (e.g., general or special purpose processors), firmware, and/or software embodied in a non-transitory computer-readable (storage) medium for execution by one or more processors to perform described functionality.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.
EC 1 is a method for refining a diagnostic machine-learning model, the method comprising: receiving, at a central computing device and from multiple point-of-care (POC) analyzers of a plurality of POC analyzers, respective signals indicative of user input approving use of the multiple POC analyzers as remote training nodes, wherein each respective POC analyzer of the plurality of POC analyzers comprises a processor and a memory storing a remote data set comprising medical data associated with patient samples analyzed by the respective POC analyzer; sending an initial training model to the multiple POC analyzers; at least some of the multiple POC analyzers training the initial training model on respective remote data sets stored on the at least some of the multiple POC analyzers using respective processors of the at least some of the multiple POC analyzers to form revised training models; receiving, at the central computing device, the revised training models from the at least some of the multiple POC analyzers; averaging, by the central computing device, parameters of the revised training models based on configurable criteria to form an adjusted training model; and sending, by the central computing device, the adjusted training model to the plurality of POC analyzers.
EC 2 is the method of EC 1, wherein the respective remote data sets remain stored on the at least some of the multiple POC analyzers during training of the initial training model.
EC 3 is the method of any of ECs 1-2, further comprising: determining whether the multiple POC analyzers are being utilized to analyze a patient sample; and in response to determining that the multiple POC analyzers are not being utilized to analyze the patient sample, the multiple POC analyzers training the initial training model.
EC 4 is the method of any of ECs 1-3, further comprising: the at least some of the multiple POC analyzers generating the respective remote data sets based on analyzing the patient samples, wherein the patient samples comprise biological samples received from a patient including tissue or blood samples.
EC 5 is the method of any of ECs 1-4, further comprising: based on any of the at least some of the multiple POC analyzers receiving a request to analyze a patient sample, interrupting training of the initial training model.
EC 6 is the method of any of ECs 1-5, further comprising: each respective POC analyzer of the plurality of POC analyzers generating the remote data set based on analyzing the patient samples, wherein the patient samples comprise biological samples received from a patient including tissue or blood samples; the central computing device querying the plurality of POC analyzers to determine an amount of data in the remote data set stored on each of the plurality of POC analyzers; and sending the initial training model to the multiple POC analyzers based on the amount of data in the remote data set stored on the multiple POC analyzers satisfying a threshold amount.
EC 7 is the method of any of ECs 1-6, wherein an integrated lab station stores paired test results of the remote data sets from the plurality of POC analyzers, and the method further comprises: the central computing device querying the integrated lab station to determine whether a paired test result exists for the remote data sets from the plurality of POC analyzers; and sending the initial training model to the multiple POC analyzers based on the multiple POC analyzers having remote data sets within the paired test result.
EC 8 is the method of any of ECs 1-7, further comprising: training the initial training model separately by the at least some of the multiple POC analyzers to form the revised training models, wherein each of the respective remote data sets stored on the at least some of the multiple POC analyzers contributes to the adjusted training model without sharing respective medical data between the plurality of POC analyzers and without sharing the respective medical data with the central computing device.
EC 9 is the method of any of ECs 1-8, further comprising: training the initial training model using federated learning via computational processing power of respective processors of the at least some of the multiple POC analyzers and via the respective remote data sets stored on the at least some of the multiple POC analyzers.
EC 10 is the method of any of ECs 1-9, further comprising: the central computing device determining which of the plurality of POC analyzers are not currently being utilized to analyze a patient sample; and the central computing device soliciting use of POC analyzers of the plurality of POC analyzers that are not currently being utilized to analyze the patient sample as respective remote training nodes.
EC 11 is the method of any of ECs 1-10, further comprising: the central computing device soliciting use of POC analyzers of the plurality of POC analyzers that are located in different geographic areas to gain access to geographically diverse remote data sets for training the initial training model.
EC 12 is the method of any of ECs 1-11, further comprising: the central computing device soliciting use of POC analyzers of the plurality of POC analyzers that are of multiple different types of POC analyzers to gain access to medically diverse remote data sets for training the initial training model.
EC 13 is the method of any of ECs 1-12, further comprising: the central computing device accessing prior stored remote data sets of the plurality of POC analyzers to determine which of the plurality of POC analyzers has complete metadata for the medical data; and the central computing device soliciting use of POC analyzers of the plurality of POC analyzers that have complete metadata for the medical data to gain access to complete remote data sets for training the initial training model.
EC 14 is the method of any of ECs 1-13, wherein the initial training model relates to an algorithm executed on medical data related to a patient of a specific species, and the method further comprises: the central computing device soliciting use of POC analyzers of the plurality of POC analyzers that are utilized to analyze patient samples of the specific species to gain access to species dependent remote data sets for training the initial training model.
EC 15 is the method of any of ECs 1-14, wherein the diagnostic machine-learning model relates to a medical condition, and the method further comprising: receiving, at the central computing device and from one of the at least some of the multiple POC analyzers that returned a respective revised training model, a notification indicating that a subsequent patient sample from a repeat patient for the medical condition has been received; sending the adjusted training model to the one of the at least some of the multiple POC analyzers that sent the notification; and retraining the adjusted training model based on an updated remote data set including medical data resulting from analysis of the subsequent patient sample.
EC 16 is the method of any of ECs 1-15, further comprising: prior to sending the adjusted training model to the plurality of POC analyzers, sending the adjusted training model to a subset of the plurality of POC analyzers to be executed within an evaluation routine running on the subset of the plurality of POC analyzers using the remote data sets stored on the subset of the plurality of POC analyzers; receiving statistics from the subset of the plurality of POC analyzers related to execution of the adjusted training model; and based on the statistics satisfying a threshold, sending the adjusted training model to all of the plurality of POC analyzers.
EC 17 is a system configured to refine a diagnostic machine-learning model, the system comprising: a plurality of POC analyzers, wherein each respective POC analyzer of the plurality of POC analyzers comprises a processor and a memory storing a remote data set comprising medical data associated with patient samples analyzed by the respective POC analyzer, and wherein each respective POC analyzer is configured to train an initial training model on respective remote data sets stored on the respective POC analyzer using a respective processor of the respective POC analyzer; a central computing device in communication with the plurality of POC analyzers, wherein the central computing device is configured to: receive from multiple point-of-care (POC) analyzers of a plurality of POC analyzers, respective signals indicative of user input approving use of the multiple POC analyzers as remote training nodes; send the initial training model to the multiple POC analyzers; receive revised training models from at least some of the multiple POC analyzers, the revised training models having been trained on respective remote data sets stored on the at least some of the multiple POC analyzers using respective processors of the at least some of the multiple POC analyzers; average parameters of the revised training models based on configurable criteria to form an adjusted training model; and send the adjusted training model to the plurality of POC analyzers.
EC 18 is the system of EC 17, wherein: the central computing device is further configured to determine whether the multiple POC analyzers are being utilized to analyze a patient sample; and in response to determining that the multiple POC analyzers are not being utilized to analyze the patient sample, the multiple POC analyzers train the initial training model.
EC 19 is the system of any of ECs 17-18, wherein: the at least some of the multiple POC analyzers generating the respective remote data sets based on analyzing the patient samples, wherein the patient samples comprise biological samples received from a patient including tissue or blood samples.
EC 20 is the system of any of ECs 17-19, wherein the central computing device is further configured to: prior to sending the adjusted training model to the plurality of POC analyzers, sending the adjusted training model to a subset of the plurality of POC analyzers to be executed within an evaluation routine running on the subset of the plurality of POC analyzers using the remote data sets stored on the subset of the plurality of POC analyzers; receiving statistics from the subset of the plurality of POC analyzers related to execution of the adjusted training model; and based on the statistics satisfying a threshold, sending the adjusted training model to all of the plurality of POC analyzers.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A method for refining a diagnostic machine-learning model, the method comprising:
receiving, at a central computing device and from multiple point-of-care (POC) analyzers of a plurality of POC analyzers, respective signals indicative of user input approving use of the multiple POC analyzers as remote training nodes, wherein each respective POC analyzer of the plurality of POC analyzers comprises a processor and a memory storing a remote data set comprising medical data associated with patient samples analyzed by the respective POC analyzer;
sending an initial training model to the multiple POC analyzers;
at least some of the multiple POC analyzers training the initial training model on respective remote data sets stored on the at least some of the multiple POC analyzers using respective processors of the at least some of the multiple POC analyzers to form revised training models;
receiving, at the central computing device, the revised training models from the at least some of the multiple POC analyzers;
averaging, by the central computing device, parameters of the revised training models based on configurable criteria to form an adjusted training model; and
sending, by the central computing device, the adjusted training model to the plurality of POC analyzers.

2. The method of claim 1, wherein the respective remote data sets remain stored on the at least some of the multiple POC analyzers during training of the initial training model.

3. The method of claim 1, further comprising:
determining whether the multiple POC analyzers are being utilized to analyze a patient sample; and
in response to determining that the multiple POC analyzers are not being utilized to analyze the patient sample, the multiple POC analyzers training the initial training model.

4. The method of claim 1, further comprising:
the at least some of the multiple POC analyzers generating the respective remote data sets based on analyzing the patient samples, wherein the patient samples comprise biological samples received from a patient including tissue or blood samples.

5. The method of claim 1, further comprising:
based on any of the at least some of the multiple POC analyzers receiving a request to analyze a patient sample, interrupting training of the initial training model.

6. The method of claim 1, further comprising:
each respective POC analyzer of the plurality of POC analyzers generating the remote data set based on analyzing the patient samples, wherein the patient samples comprise biological samples received from a patient including tissue or blood samples;
the central computing device querying the plurality of POC analyzers to determine an amount of data in the remote data set stored on each of the plurality of POC analyzers; and
sending the initial training model to the multiple POC analyzers based on the amount of data in the remote data set stored on the multiple POC analyzers satisfying a threshold amount.

7. The method of claim 1, wherein an integrated lab station stores paired test results of the remote data sets from the plurality of POC analyzers, and the method further comprises:
the central computing device querying the integrated lab station to determine whether a paired test result exists for the remote data sets from the plurality of POC analyzers; and
sending the initial training model to the multiple POC analyzers based on the multiple POC analyzers having remote data sets within the paired test result.

8. The method of claim 1, further comprising:
training the initial training model separately by the at least some of the multiple POC analyzers to form the revised training models,
wherein each of the respective remote data sets stored on the at least some of the multiple POC analyzers contributes to the adjusted training model without sharing respective medical data between the plurality of POC analyzers and without sharing the respective medical data with the central computing device.

9. The method of claim 1, further comprising:
training the initial training model using federated learning via computational processing power of respective processors of the at least some of the multiple POC analyzers and via the respective remote data sets stored on the at least some of the multiple POC analyzers.

10. The method of claim 1, further comprising:
the central computing device determining which of the plurality of POC analyzers are not currently being utilized to analyze a patient sample; and
the central computing device soliciting use of POC analyzers of the plurality of POC analyzers that are not currently being utilized to analyze the patient sample as respective remote training nodes.

11. The method of claim 1, further comprising:
the central computing device soliciting use of POC analyzers of the plurality of POC analyzers that are located in different geographic areas to gain access to geographically diverse remote data sets for training the initial training model.

12. The method of claim 1, further comprising:
the central computing device soliciting use of POC analyzers of the plurality of POC analyzers that are of multiple different types of POC analyzers to gain access to medically diverse remote data sets for training the initial training model.

13. The method of claim 1, further comprising:
the central computing device accessing prior stored remote data sets of the plurality of POC analyzers to determine which of the plurality of POC analyzers has complete metadata for the medical data; and
the central computing device soliciting use of POC analyzers of the plurality of POC analyzers that have complete metadata for the medical data to gain access to complete remote data sets for training the initial training model.

14. The method of claim 1, wherein the initial training model relates to an algorithm executed on medical data related to a patient of a specific species, and the method further comprises:
the central computing device soliciting use of POC analyzers of the plurality of POC analyzers that are utilized to analyze patient samples of the specific species to gain access to species dependent remote data sets for training the initial training model.

15. The method of claim 1, wherein the diagnostic machine-learning model relates to a medical condition, and the method further comprising:
receiving, at the central computing device and from one of the at least some of the multiple POC analyzers that returned a respective revised training model, a notification indicating that a subsequent patient sample from a repeat patient for the medical condition has been received;
sending the adjusted training model to the one of the at least some of the multiple POC analyzers that sent the notification; and
retraining the adjusted training model based on an updated remote data set including medical data resulting from analysis of the subsequent patient sample.

16. The method of claim 1, further comprising:
prior to sending the adjusted training model to the plurality of POC analyzers, sending the adjusted training model to a subset of the plurality of POC analyzers to be executed within an evaluation routine running on the subset of the plurality of POC analyzers using the remote data sets stored on the subset of the plurality of POC analyzers;
receiving statistics from the subset of the plurality of POC analyzers related to execution of the adjusted training model; and
based on the statistics satisfying a threshold, sending the adjusted training model to all of the plurality of POC analyzers.

17. A system configured to refine a diagnostic machine-learning model, the system comprising:
a plurality of POC analyzers, wherein each respective POC analyzer of the plurality of POC analyzers comprises a processor and a memory storing a remote data set comprising medical data associated with patient samples analyzed by the respective POC analyzer, and wherein each respective POC analyzer is configured to train an initial training model on respective remote data sets stored on the respective POC analyzer using a respective processor of the respective POC analyzer;
a central computing device in communication with the plurality of POC analyzers, wherein the central computing device is configured to:
receive from multiple point-of-care (POC) analyzers of a plurality of POC analyzers, respective signals indicative of user input approving use of the multiple POC analyzers as remote training nodes;
send the initial training model to the multiple POC analyzers;
receive revised training models from at least some of the multiple POC analyzers, the revised training models having been trained on respective remote data sets stored on the at least some of the multiple POC analyzers using respective processors of the at least some of the multiple POC analyzers;
average parameters of the revised training models based on configurable criteria to form an adjusted training model; and
send the adjusted training model to the plurality of POC analyzers.

18. The system of claim 17, wherein:
the central computing device is further configured to determine whether the multiple POC analyzers are being utilized to analyze a patient sample; and
in response to determining that the multiple POC analyzers are not being utilized to analyze the patient sample, the multiple POC analyzers train the initial training model.

19. The system of claim 17, wherein:
the at least some of the multiple POC analyzers generating the respective remote data sets based on analyzing the patient samples, wherein the patient samples comprise biological samples received from a patient including tissue or blood samples.

20. The system of claim 17, wherein the central computing device is further configured to:
prior to sending the adjusted training model to the plurality of POC analyzers, sending the adjusted training model to a subset of the plurality of POC analyzers to be executed within an evaluation routine running on the subset of the plurality of POC analyzers using the remote data sets stored on the subset of the plurality of POC analyzers;
receiving statistics from the subset of the plurality of POC analyzers related to execution of the adjusted training model; and
based on the statistics satisfying a threshold, sending the adjusted training model to all of the plurality of POC analyzers.
